(19) United States
(12) Patent Application Publication
(10) Pub. No.: US 2025/0352467 A1
(43) Pub. Date: Nov. 20, 2025

(54) PHARMACEUTICAL COMPOSITION FOR DRY POWDER INHALATION AND PREPARATION METHOD THEREOF

(71) Applicant: ASG INSPIRATION LABORATORY (SINGAPORE) PTE. LTD., SINGAPORE (SG)

(72) Inventors: Sung Tsai YU, Taipei City (TW); Johnson LEE, Taipei City (TW); Hao-Wei HUANG, Taipei City (TW); Hsin-Ke LI, Taipei City (TW); Shih-Tan CHEN, Taipei City (TW); Hou In LEONG, Taipei City (TW); Jao Juen HUNG, Taipei City (TW); Shih Pan CHAO, Taipei City (TW)

(21) Appl. No.: 19/203,219

(22) Filed: May 9, 2025

Related U.S. Application Data
(60) Provisional application No. 63/648,163, filed on May 15, 2024.

(51) Int. Cl. A61K 9/00; A61K 9/16; B82Y 5/00

(52) U.S. Cl. CPC A61K 9/0075; A61K 9/1623; B82Y 5/00

(57) ABSTRACT

A pharmaceutical composition for dry powder inhalation is provided, including an active ingredient and a pharmacologically acceptable excipient. The active ingredient includes an active compound with a molecular weight of less than 1000 Daltons. The pharmacologically acceptable excipient includes amino acid, sugar, sugar alcohol, polyol, phospholipid, or a combination thereof, or a combination thereof, in which a weight ratio of the pharmacologically acceptable excipient and the active ingredient is from 1:1 to 99:1. In some embodiments of the present disclosure, a method of preparing a pharmaceutical composition for dry powder inhalation is further provided. By regulating the amount of the pharmacologically acceptable excipient, the aerosol property of the pharmaceutical composition can be predominantly influenced by the pharmacologically acceptable excipient, thereby reducing the impact of the active ingredient on the aerosol property and expanding the range of the active ingredient applicable to the pharmaceutical composition.

### CROSS-REFERENCE TO RELATED APPLICATION

This application claims priority to U.S. Provisional Patent Application No. 63/648,163, filed May 15, 2024, the disclosure of which is hereby incorporated by reference in its entirety.

### BACKGROUND

### Field of Invention

The present disclosure relates to a pharmaceutical composition for dry powder inhalation and a preparation thereof. In particular, the present disclosure relates to a pharmaceutical composition that can be widely applied to various active ingredients.

### Description of Related Art

Dry powder used in dry powder inhalers (DPI) is primarily either a standalone active pharmaceutical ingredient (API) or a formulation in which the proportion of API is significantly higher than that of the excipient. Consequently, the physical properties of the microparticute particles in the dry powder are predominantly determined by the physical properties of API, thereby necessitating multiple experimental iterations in practice, relying on a trial-and-error approach to identify relatively optimal formulation conditions.

However, due to variations in APIs, re-evaluating the formulation conditions (particularly the tests of aerosol properties during the preparation process of generic drugs) to determine the dry powder formulation leads to prolonged development timelines and increased research and development costs.

Therefore, how to provide a pharmaceutical composition for dry powder inhalation to reduce formula testing time and development cost remains to be solved.

### SUMMARY

In one aspect of the present disclosure, a pharmaceutical composition for dry powder inhalation is provided, including: an active ingredient and a pharmacologically acceptable excipient. The active ingredient includes an active compound with a molecular weight of less than 1000 Daltons. The pharmacologically acceptable excipient includes amino acid, sugar, sugar alcohol, polyol, phospholipid, or a combination thereof, in which a weight ratio of the pharmacologically acceptable excipient and the active ingredient is from 1:1 to 99:1.

In some embodiments, a weight percentage of the active ingredient is from 1% to 50% and a weight percentage of the pharmacologically acceptable excipient is from 50% to 99% based on 100% by weight of the active ingredient and the pharmacologically acceptable excipient.

In some embodiments, the active compound includes organic compound.

In some embodiments, the amino acid includes glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline or a combination thereof.

In some embodiments, the sugar includes disaccharide or polysaccharide.

In some embodiments, the disaccharide includes sucrose, trehalose, lactose, maltose, or a combination thereof.

In some embodiments, the polysaccharide includes chitosan, chitosan salt, chitosan glutamate, hyaluronic acid, starch, or a combination thereof.

In some embodiments, the sugar alcohol includes glucose alcohol, xylitol, sorbitol, maltitol, erythritol, lactitol, mannitol, or a combination thereof.

In some embodiments, the polyol includes polyvinyl alcohol, polyethylene glycol, or a combination thereof.

In some embodiments, the phospholipid includes dipalmitoyl phosphatidylcholine (DPPC), distearoyl phosphatidyl choline (DSPC) or a combination thereof.

In some embodiments, the active ingredient and the pharmacologically acceptable excipient forms a microparticulate particle having a particle size of from 50 nm to 5 μm.

In some embodiments, the microparticulate particle is provided in a spherical-shaped form, a solid polyhedron form, or a combination thereof.

In some embodiments, the pharmacologically acceptable excipient includes a first pharmacologically acceptable excipient and a second pharmacologically acceptable excipient different from the first pharmacologically acceptable excipient.

In some embodiments, a weight percentage of the first pharmacologically acceptable excipient and the second pharmacologically acceptable excipient is from 1:1 to 99:1 when the first pharmacologically acceptable excipient is the amino acid, and the second pharmacologically acceptable excipient is the sugar or the sugar alcohol.

In another aspect of the present disclosure, a method of preparing a pharmaceutical composition for dry powder inhalation is provided, including: dissolving an active ingredient and a pharmacologically acceptable excipient in a solvent to form a mixture, in which the active ingredient includes an active compound with a molecular weight of less than 1000 Daltons, and the pharmacologically acceptable excipient includes amino acid, sugar, sugar alcohol, polyol, phospholipid, or a combination thereof, in which a weight ratio of the pharmacologically acceptable excipient and the active ingredient is from 1:1 to 99:1; and spray drying the mixture to form a pharmaceutical composition in a microparticulate form.

In some embodiments, dissolving the active ingredient and the pharmacologically acceptable excipient includes dissolving the active ingredient, the first pharmacologically acceptable excipient and the second pharmacologically acceptable excipient in the solvent, in which the second pharmacologically acceptable excipient is different from the first pharmacologically acceptable excipient.

In some embodiments, a weight percentage of the first pharmacologically acceptable excipient and the second pharmacologically acceptable excipient is from 1:1 to 1:99 when the first pharmacologically acceptable excipient is the amino acid, and the second pharmacologically acceptable excipient is the sugar or the sugar alcohol.

In some embodiments, the solvent includes water, ethanol, methanol, dichloromethane, ethyl acetate, acetonitrile, acetone, dimethyl sulfoxide, or a combination thereof.

In some embodiments, a weight percentage of the active ingredient and the pharmacologically acceptable excipient is from 0.2% to 3% based on 100% by weight of the mixture.

In some embodiments, spray drying the mixture is performed at an outlet temperature of from 35° C. to 110° C.

### BRIEF DESCRIPTION OF THE DRAWINGS

In order to make the above-mentioned and other objects, features, advantages and embodiments of the present disclosure more clearly understood, descriptions of accompanying drawings are as follows:

FIG. 1 illustrates a flow chart of a method of preparing a pharmaceutical composition for dry powder inhalation in some embodiments of the present disclosure.

FIG. 2 is an electron microscopy image, representing microparticulate particles (composition: vardenafil+leucine+mannitol) under the field of view of an electron microscope.

FIG. 3 illustrates a comparison chart of aerosol properties of group Var only (vardenafil), group Var+Man (vardenafil+mannitol) and group Var+Leu+Man (vardenafil+leucine+mannitol) detected by using Next Generation Impactor (NGI).

FIG. 4 illustrates a comparison chart of aerosol properties of group Var+Leu+Tre (vardenafil+leucine+trehalose dihydrate) and group Bed+Leu+Tre (bedaquiline+leucine+trehalose dehydrate).

### DETAILED DESCRIPTION

In order that the present disclosure is described in detail and completeness, implementation aspects and specific embodiments of the present disclosure with illustrative description are presented, but those are not the only form for implementation or plucuse of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein may include a plurality of sub-steps or actions.

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, “active ingredient” refers to a chemical substance or a combination of multiple chemical substances used for the treatment, prevention of diseases, or alleviation of pain.

As used herein, “pharmacologically acceptable excipient” refers to pharmaceutical additives without pharmacological activity and used in pharmaceutical compositions according to different purposes and functions.

The commonly known dry powder containing an active ingredient primarily uses the active ingredient as the main component. However, since different active ingredients typically exhibit different physicochemical properties due to structural differences, it becomes necessary to conduct optimization condition tests for each dry powder when the active ingredient varies. Through a trial-and-error process, the appropriate types of excipients and the formulation of components are determined for dry powders with different active ingredients. However, this approach (trial-and-error process) results in a prolonged research and development process and significant expenditure of research and development funds.

The primary objective of the present disclosure is to provide a pharmaceutical composition for dry powder inhalation using the pharmacologically acceptable excipient as the main component. By designing the aerosol property or aerodynamic (e.g., enhancing the fine particle fraction (FPF)) to be governed by the pharmacologically acceptable excipient, a formulation platform compatible with various active ingredients is established. Through the selection of specific type of the pharmacologically acceptable excipient and increasing its content to a predominant proportion, the influences of different active ingredients on the aerosol property of the dry powder is minimized.

Therefore, a single formulation to be applicable to multiple active ingredients is allowed, enabling dry powders with different active ingredients to exhibit similar aerosol properties. Consequently, the development timeline and costs associated with simultaneously developing multiple dry powders for different active ingredients are significantly reduced.

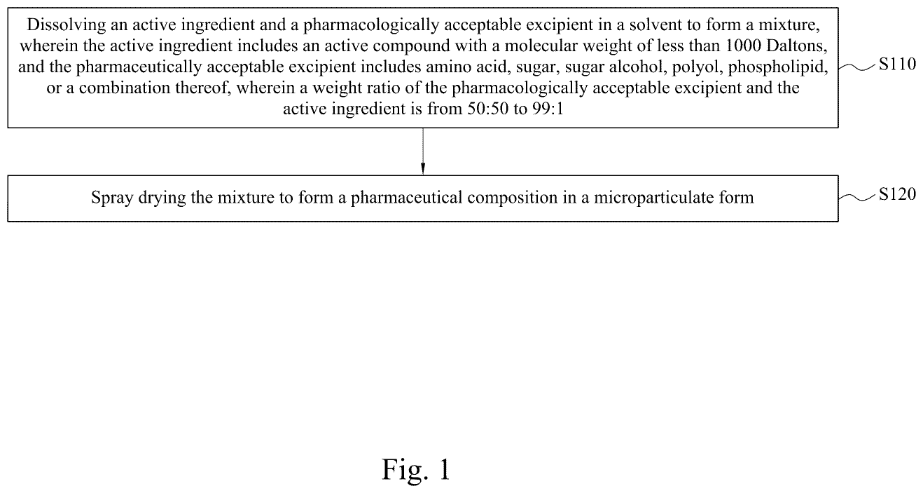

Please refer to FIG. 1, representing a flow chart of a method 100 of preparing a pharmaceutical composition for dry powder inhalation in some embodiments of the present disclosure, including step S110 and step S120. It should be emphasized that, compared with the use of the active ingredient alone (including both unspray-dried and spray-dried forms), by mixing a pharmacologically acceptable excipient with an active ingredient in a mixture, and subsequently spray drying the mixture, the shape and particle size of a microparticulate particle can be modulated (including but not limited to shapes such as spherical or polyhedral forms and more consistent particle sizes across different batches), thereby improving the aerosol property and onset time of the microparticulate particle.

First, please refer to step S110, an active ingredient and a pharmacologically acceptable excipient are dissolved in a solvent to form a mixture, wherein the active ingredient includes an active compound with a molecular weight of less than 1000 Daltons, and the pharmacologically acceptable excipient includes amino acid, sugar, sugar alcohol, polyol, phospholipid, or a combination thereof, wherein a weight ratio of the pharmacologically acceptable excipient and the active ingredient is from 50:50 (1:1) to 99:1.

In some embodiments, the active ingredient is an active compound with a molecular weight of less than 1000 Daltons, and the active compound, such as small molecule drug or a pharmaceutically acceptable salt thereof, including but not limited to salts, esters, complexes, chelating agents, cage compounds, racemates, mirror image isomers, or the like. In some embodiments, the active compound includes organic compound, such as phosphodiesterase type 5 inhibitor (PDE5-I), antituberculosis drug, analgesic drug, expectorant drug, or a combination thereof. PDE5-I includes sildenafil, vardenafil, tadalafil, avanafil, or pharmacologically acceptable salt thereof. Antituberculosis drug includes bedaquiline, isoniazid, rifampin, ethambutol, pyrazinamide, rifabutin, or a pharmacologically acceptable salt thereof. Analgesic drug includes acetaminophen, aspirin, or a pharmacologically acceptable salt thereof. Expectorant drug includes erdosteine, bromhexine, ambroxol, or a pharmacologically acceptable salt thereof.

In some embodiments, the active ingredient includes a single active ingredient or multiple types of active ingredients (e.g., the first active ingredient and the second active ingredient). Since the aerosol property of the pharmaceutical composition is primarily governed by the pharmacologically acceptable excipient, it can be expected that even if the pharmaceutical composition carries multiple active ingredients, it will exhibit aerosol property similar to that of a pharmaceutical composition carrying a single active ingredient.

In some embodiments, the solvent includes water, ethanol, methanol, dichloromethane, ethyl acetate, acetonitrile, acetone, dimethyl sulfoxide, or a combination thereof.

In some embodiments, a weight ratio of the pharmacologically acceptable excipient and the active ingredient is from 1:1 to 99:1, such as 1:1, 10:1, 20:1, 30:1, 40: 1, 50:1, 60:1, 70:1, 80:1, 90:1, 95:1, 99:1, or any value between any interval of the abovementioned values. If the weight ratio is too low, the pharmaceutical composition is hardly regulated by the pharmacologically acceptable excipient, and the aerosol property of the pharmaceutical composition will be easily influenced by the properties of different active ingredients, making it challenge to achieve consistent results across various active ingredients. If the weight ratio is excessively high, the content of the active ingredient becomes too low, requiring users to consume an excessively large amount of the pharmaceutical composition to meet the required dosage.

In some embodiments, a viscosity of the pharmacologically acceptable excipient is lower than 3 dl/g, such as from 0.1 dl/g to 3 dl/g. If the viscosity is too high, the pharmacologically acceptable excipient is hardly spray dried and the particle size of the microparticulate particle is too big to meet the requirement of inhalation administration.

In some embodiments, the amino acid includes glycine, alanine, valine, leucine, isoleucine, phenylalanine, tryptophan, tyrosine, aspartic acid, histidine, asparagine, glutamic acid, lysine, glutamine, glutamic acid, methionine, arginine, serine, threonine, cysteine, proline or a combination thereof.

In some embodiments, the sugar includes disaccharide or polysaccharide, and the term “sugar” herein encompasses hydrated forms. In some embodiments, the disaccharide includes sucrose, trehalose, lactose, maltose, or a combination thereof. In some embodiments, the polysaccharide includes chitosan, chitosan salt, chitosan glutamate, hyaluronic acid, starch, or a combination thereof. In some embodiments, the sugar alcohol includes glucose alcohol, xylitol, sorbitol, maltitol, erythritol, lactitol, mannitol, or a combination thereof. In some embodiments, the polyol includes polyvinyl alcohol, polyethylene glycol, or a combination thereof. In some embodiments, the phospholipid includes dipalmitoyl phosphatidylcholine, distearoyl phosphatidyl choline, or a combination thereof.

In some embodiments, the pharmacologically acceptable excipient includes single or multiple types. In some embodiments, multiple types of the pharmacologically acceptable excipient includes such as a first pharmacologically acceptable excipient and a second pharmacologically acceptable excipient different from the first pharmacologically acceptable excipient or includes more than three types of the pharmacologically acceptable excipient. Typically, people skilled in the art can adjust the types and concentration of the pharmacologically acceptable excipient according to the requirements of aerosol property.

In some embodiments, a weight percentage of the first pharmacologically acceptable excipient and the second pharmacologically acceptable excipient is from 1:1 to 1:99, such as 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:99 or any value between any interval of the abovementioned values when the first pharmacologically acceptable excipient is the amino acid, and the second pharmacologically acceptable excipient is the sugar (such as trehalose belonging to disaccharide) or the sugar alcohol (such as mannitol). When the weight ratio is too low (with a relatively high amino acid content), the proportion of irregular shapes increases, and the stability of aerosol property decreases.

It can be understood that, compared with the active ingredient, the pharmacologically acceptable excipient is generally more prone to dissolving; therefore, in the mixture with a high proportion of the pharmacologically acceptable excipient, the pharmacologically acceptable excipient and the active ingredient can be directly mixed in the solvent to dissolve the respective components. In some embodiments, when the pharmacologically acceptable excipient is multiple types, dissolving the active ingredient and the pharmacologically acceptable excipient includes dissolving the active ingredient, a first pharmacologically acceptable excipient and a second pharmacologically acceptable excipient in the solvent.

In some other embodiments, to further ensure that all components are properly dissolving and to prevent aggregation, the pharmacologically acceptable excipient and the active ingredient can be separately dissolved in the first solvent and the second solvent, respectively, following by combining into the mixture.

In some embodiments, a weight percentage of the active ingredient and the pharmacologically acceptable excipient is from 0.2% to 3%, such as 0.2%, 0.3%, 0.4%, 0.5%, 1%, 1.5%, 2%, 2.5%, 3% or any value between any interval of the abovementioned values, based on 100% by weight of the mixture. If the weight percentage is too low, the yield of spray drying is limited. If the weight percentage is too high, the mixture may become uneven and excessively viscous, making it unsuitable for spray drying, thereby limiting the efficiency of spray drying.

Please refer to step S120, the mixture is spray dried to form a pharmaceutical composition in a microparticulate form.

In some embodiments, spray drying the mixture is performed at an outlet temperature of from 35° C. to 110° C., such as 35° C., 40° C., 45° C., 50° C., 55° C., 60° C., 65° C., 70° C., 75° C., 80° C., 85° C., 90° C., 95° C., 100° C., 110° C. or any value between any interval of the abovementioned values. If the outlet temperature is too low, the sprayed droplets are too large, the particle size of the sprayed particles tends to be larger, and the shape of the sprayed particles will be difficult to maintain in a spherical-shaped form or polyhedron and tend to be irregular. If the outlet temperature is high, the structure of the active ingredient or the pharmacologically acceptable excipient may be changed, thereby influencing the functions.

In some embodiments, a weight percentage of the active ingredient is from 1% to 50% (such as 1%, 10%, 20%, 30%, 40%, 50% or any value between any interval of the abovementioned values) and a weight percentage of the pharmacologically acceptable excipient is from 50% to 99% (such as 50%, 60%, 70%, 80%, 90%, 95%, 99% or any value between any interval of the abovementioned values) based on 100% by weight of the active ingredient and the pharmacologically acceptable excipient. If the weight percentage of the active ingredient is too low or the weight percentage of the pharmacologically acceptable excipient is too high, the active ingredient provided by the pharmaceutical composition of a specific unit is restricted. If the weight percentage of the active ingredient is too high or the weight percentage of the pharmacologically acceptable excipient is too low, the extent to which the aerosol property of the pharmaceutical composition is influenced by the active ingredient increases. If the original active ingredient is replaced with another active ingredient, the degree of change in the aerosol property will become more pronounced, making it difficult for the pharmaceutical composition to serve as a universal platform for different active ingredients.

In some embodiments, the active ingredient and the pharmacologically acceptable excipient forms a microparticulate particle having a particle size of from 50 nm to 5 μm, such as 50 nm, 100 nm, 500 nm, 1 μm, 2 μm, 3 μm, 4 μm, 5 μm or any value between any interval of the abovementioned values. It is noteworthy that the particle size of the microparticulate particle is smaller than that of the active ingredient without spray drying. By establishing the particle size range of the microparticulate particle, the aerosol property can be regulated to meet the particle size requirements suitable for pulmonary inhalation.

In some embodiments, the microparticulate particle is provided in a solid spherical-shaped form, a solid polyhedron form, or a combination thereof. Compared with the irregular shape formed by no spray drying, the shape of the microparticulate particle can increase the flight distance of the microparticulate particle and enhance the distribution ratio in lungs while administration.

In some embodiments, the method 100 further includes: testing the physical properties (e.g., particle size distribution, aerodynamic particle size distribution analysis, surface area, surface electrostatic forces, etc.) or human test data of the dry powder prepared using various formulations of active ingredients and pharmacologically acceptable excipients to obtain a test dataset; employing machine ensemble learning, artificial neural networks, or a combination of both on the test dataset for analysis and processing to establish a simulation model. The simulation model enables the prediction of aerosol property or the pharmacokinetic performance in the human body of dry powders when using different active ingredients or varying formulation ratios. Through the analytical processing of machine learning or neural networks, the time required for conditional testing can be further reduced, and more accurate predictions regarding the characteristics of dry powder can be achieved. In some embodiments, machine ensemble learning includes such as Boosting, Adaptive Boosting (AdaBoost), Gradient Boosting Decision Tree (GBDT), eXtreme Gradient Boosting (XGboost), or a combination thereof. In some embodiments, neural networks include such as Artificial Neural Network (ANN), convolutional neural network (CNN), or a combination thereof.

In some embodiments, a microparticulate particle can be packed into capsules, aluminum foil blisters, and drug storage tanks in dry powder inhalation devices for subjects in need to inhale.

In some embodiments, the method 100 further includes mixing the microparticulate particle with a flavoring agent (such as menthol or natural flavors tasting like lemon, strawberry, orange, etc.) of a weight percentage of less than 1% (such as 0.1%, 0.5%, 1% or any value between any interval of the abovementioned values) to reduce bitter taste when inhalation. In one embodiment, mixing the microparticulate particle with the flavoring agent of the weight percentage of 1% has better efficiency for reducing bitter taste.

It should be understood that the above-described embodiments and the following examples are given by way of illustration, not limitation. Various changes and modifications within the scope of the present invention will become apparent to those skilled in the art from the present description.

For clarifying the pharmaceutical composition for dry powder inhalation and preparation method thereof, several examples and functional testes are described below in sequence.

### Example 1—Preparation Method of Pharmaceutical Composition for Dry Powder Inhalation and Physical Properties

### 1. Preparation Method

Step a: Vardenafil (also abbreviated as Var) and two types of the pharmacologically acceptable excipients (leucine (also abbreviated as Leu) and mannitol (also abbreviated as Man) being selected here) were mixed according to the formulation in Table 1 and dissolved in the solvent containing 20% ethanol to obtain a mixture, in which the weight ratio of mannitol and leucine was 5.6:1.

TABLE 1

| Weight Ratio of Pharmacologically | |
|---|---|
| Acceptable Excipient (Leucine + Mannitol) | |
| and Vardenafil | Concentration of Vardenafil, Leucine and |
| (w:w) | Mannitol in the Mixture (w/v, %) |
| 95:5 | 0.5 |
| 97:3 | |
| 99:1 | |

Step b: The mixture obtained from step a was spray dried form a microparticulate particle under the following conditions: dry gas flow rate of 25 m³/hr, flow rate of spray gas of 1000 L/hr, feeding rate of 5 mL/minute, inlet temperature of 100° C. and outlet temperature of 50° C.

Compared with other conditions in Table 1, the microparticulate particles prepared by the condition of the weight ratio of 99:1 and the concentration of 0.5% had the smaller particle size and more spherical-shaped form and were selected for the following physical observations.

### Shape of Microparticulate Particle

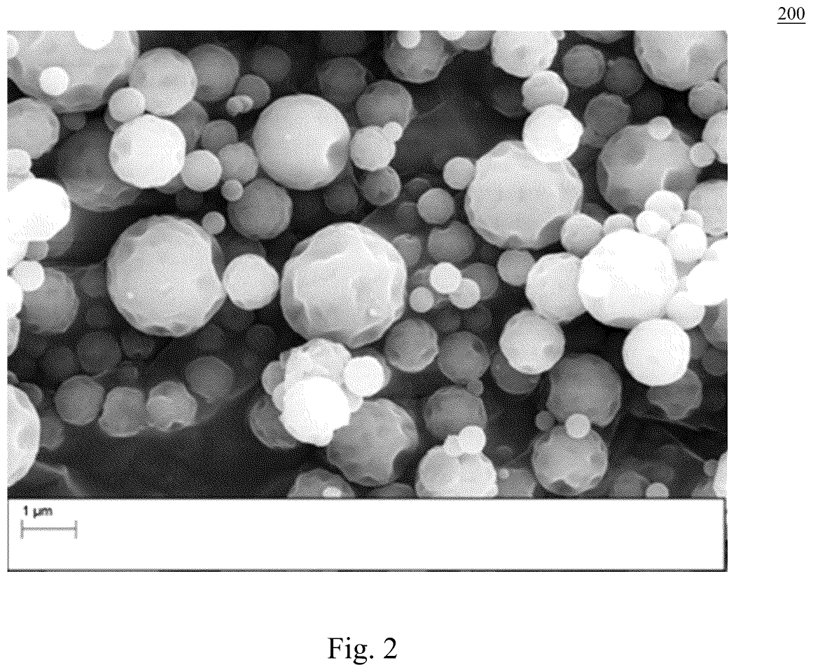

Appearances of dry powders (vardenafil+leucine+mannitol) were observed under the field of an electron microscope, please refer to FIG. 2 (electron microscopy image 200) for the result, representing microparticulate particles obtained by spray drying vardenafil, leucine and mannitol.

FIG. 2 represented that the shape of microparticulate particles obtained by spray drying was spherical-shaped form or polyhedral form (such as golf ball type, that is, solid polyhedron form that had multiple recesses on the surface). Furthermore, although not illustrated in figures, it should be noted that compared with vardenafil without spray drying, the microparticulate particles obtained by spray drying the pharmacologically acceptable excipient and vardenafil together exhibited a more uniform shape.

### Particle Size

The particle size (D10, D50, and D90) of the microparticulate particles (vardenafil+leucine+mannitol) was analyzed by using a particle size analyzer and summarized in Table 2.

TABLE 2

| | D10 | D50 | D90 |
|---|---|---|---|
| Particle Size (μm) | 0.78 | 1.59 | 3.41 |

Note: D10 referred to the particle size corresponding to the cumulative frequency of 10%. D50 referred to the particle size corresponding to the cumulative frequency of 50%. D90 referred to the particle size corresponding to the cumulative frequency of 90%.

As shown in Table 2, more than 90% of the microparticulate particles had a particle size of less than 4 μm, D50 was only 1 μm to 2 μm, and D10 was only 0.5 μm to 0.9 μm, which were smaller than those of vardenafil without spray drying (the vardenafil values were omitted and not shown in Table 2).

### Example 2—Comparison of Aerosol Property

1. Comparison Between Dry Powder Prepared by Directly Spray Drying Active Ingredient, Dry Powder Prepared with One Pharmacologically Acceptable Excipient and Dry Powder Prepared with Multiple Pharmacologically Acceptable Excipients

For compare whether the aerosol property of the dry powder prepared by adding pharmacologically acceptable excipient or adding multiple pharmacologically acceptable excipients was influenced, the microparticulate particles of group Var only, group Var+Man and group Var+Leu+Man were provided for detecting aerosol property according to cascade impaction (CI) by using Next Generation Impactor (NGI) (brand: Copley Scientific, device name: Model 170) at a flow rate of 60 L/min. Group Var only was prepared by directly spray drying vardenafil, group Var+Man was prepared by spray drying the mixture containing vardenafil and mannitol, in which the weight percentage of vardenafil in the microparticulate particle was 1%, and the weight percentage of mannitol was 99%; group Var+Leu+Man was prepared by spray drying the mixture containing vardenafil, leucine and mannitol, in which the weight percentage of vardenafil in the microparticulate particle was 1%, the weight percentage of leucine in the microparticulate particle was 15%, and the weight percentage of mannitol in the microparticulate particle was 84%.

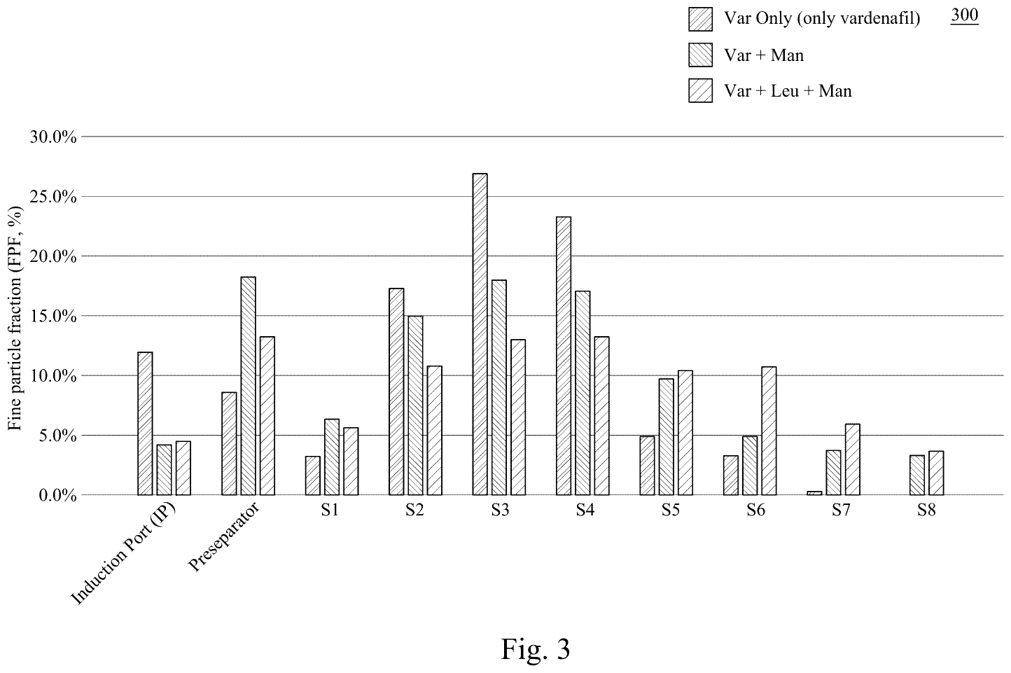

Please refer to FIG. 3, in which fine particle fraction (FPF) was the percentage of the cumulative deposition content of the microparticulate particles smaller than 5 μm to the total output content of the microparticulate particles, which reflected the effective deposition ratio of the microparticulate particles in the lungs.

FIG. 3 (aerosol property comparison FIG. 300) represented that compared with group Var only, the microparticulate particles of group Var+Man and group Var+Leu+Man were less distributed in the front stages (such as the stages of IP and S2 to S4) (the relatively shorter flight distance) and increasingly distributed in the following stages, such as the stages of S5 to S8, indicating the relatively longer flight distance was achieved. That is, compared with group Var only, group Var+Man and group Var+Leu+Man demonstrated the longer flight distance.

Furthermore, compared with group Var+Man, it was observed that the microparticulate particles of group Var+Leu+Man that included two different types of the pharmacologically acceptable excipients were increasingly distributed in the stages at stage S5 to stage of S8, the stages that required the relatively longer flight distance. That is, compared with group Var+Man, group Var+Leu+Man further demonstrated the longer flight distance. Therefore, FIG. 3 indicated that the ranking of the flight distance (long to short) among the microparticulate particles of the three groups was (1) the microparticulate particles prepared by adding two different types of the pharmacologically acceptable excipients (leucine and mannitol) before spray drying, (2) the microparticulate particles prepared by adding one type of the pharmacologically acceptable excipient before spray drying, and (3) the microparticulate particles prepared by directly spray drying vardenafil in order. That is, according to the data above, it was demonstrated that the microparticulate particles prepared by adding the pharmacologically acceptable excipient achieved the higher distribution ratio in lungs than the microparticulate particles prepared by directly spray drying. Furthermore, the microparticulate particles prepared by adding multiple different types of the pharmacologically acceptable excipients (such as leucine and mannitol) achieved the higher distribution ratio in lungs than the microparticulate particles prepared by adding only one type of the pharmacologically acceptable excipient (such as mannitol).

### 2. Comparison of Dry Powders Having Different Active Ingredients

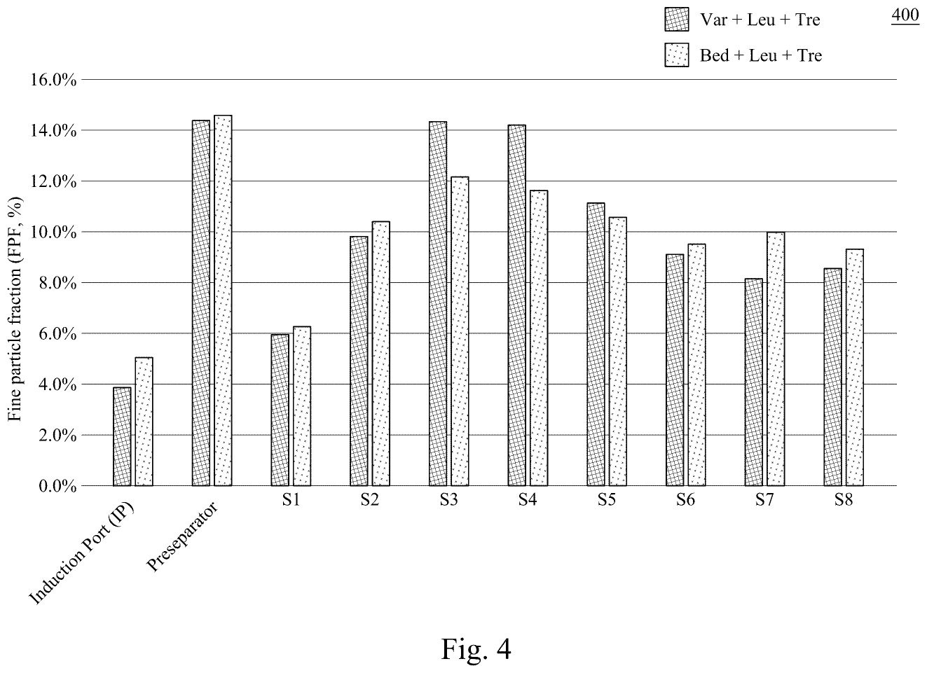

In order to verify whether the use of the pharmacologically acceptable excipient as the formulation matrix can effectively reduce the impact of the active ingredient on the aerosol property of the microparticulate particle, thereby creating a dry powder formulation platform applicable to various active ingredients, the microparticulate particles of group Var+Leu+Tre and group Bed+Leu+Tre were prepared via the process similar to that described in Example 1 for the purpose of evaluating aerosol property. Furthermore, according to the experimental method similar to point 1 of Example 2, the aerosol properties of two groups of dry powder were analyzed, and the results were summarized in FIG. 4 and Table 3. Group Var+Leu+Tre was obtained by spray drying the mixture containing vardenafil, leucine, and trehalose dihydrate (hereinafter abbreviated as tre here), where leucine and trehalose dihydrate served as the pharmacologically acceptable excipient. The weight percentage of vardenafil in the microparticulate particle was 1%, the weight percentage of leucine was 15%, and the weight percentage of trehalose dihydrate was 84%. Group Bed+Leu+Tre was obtained by spray drying the mixture containing bedaquiline (hereinafter abbreviated as Bed here), leucine, and trehalose dihydrate, where the weight percentage of bedaquiline in the microparticulate particles was 1%, the weight percentage of leucine was 15%, and the weight percentage of trehalose dihydrate was 84%.

As observed in FIG. 4 (aerosol property comparison FIG. 400), group Var+Leu+Tre and group Bed+Leu+Tre exhibited a similar distribution trend of dry powder.

TABLE 3

| | | | | Geometric |
|---|---|---|---|---|
| Analysis Result of | | | MMAD | Standard |
| NGI | Emitted Dose (%) | FPF (%) | (μm) | Deviation |
| Var + Leu + Tre | 83.62 | 71.42 | 1.35 | 1.52 |
| Bed + Leu + Tre | 84.79 | 68.02 | 1.12 | 1.63 |

### Note

Emitted dose referred to the weight percentage of the active ingredient in the dry powder actually released in the NGI, relative to the total amount of the active ingredient in the initial dry powder.
Fine Particle Fraction (FPF) referred to the weight percentage of the cumulative deposition of microparticulate particles smaller than 5 μm within the total content of microparticulate particles released (emitted dose).
MMAD referred to mass median aerodynamic diameter.
Geometric standard deviation (GSD) was defined as the square root of the ratio of the aerodynamic diameter at 84% cumulative mass to that at 16% cumulative mass, representing the dispersity (or variability) extent of particle size distribution of dry powder.

The results represented in Table 3 indicated that even with different active ingredients, the aerosol property-related parameters of group Var+Leu+Tre and group Bed+Leu+Tre were similar, revealing that the two dry powders exhibited comparable aerosol properties.

Based on the results of FIG. 4 and Table 3, it was concluded that even when different active ingredients were selected, the proportion of the pharmacologically acceptable excipient was increased to a high level (e.g., 99% mannitol, 15% leucine+84% mannitol, or 15% leucine+84% trehalose dihydrate in the aforementioned experiments) lead to a scenario where the content of the pharmacologically acceptable excipient dominated. Consequently, the influence of the active ingredient on aerosol property became minimal, and the aerosol property of the microparticulate particles were primarily governed by the pharmacologically acceptable excipient.

That is, by designing with a relatively large proportion of a specifically selected pharmacologically acceptable excipient, the influence of the active ingredient on the performance of the dry powder was reduced. This approach established a formulation platform compatible with various active ingredients, enabling the dry powders containing different active ingredients to exhibit similar aerosol property. Consequently, this method reduced the trial-and-error process traditionally required for preparing the dry powders (where the active ingredient predominantly determined the aerosol properties) tailored to different active ingredients.

Additionally, it was worth emphasizing that further cross-comparison between group Var+Leu+Man in FIG. 3 and group Var+Leu+Tre in FIG. 4 (the difference between the two groups being that mannitol in group Var+Leu+Man was replaced with trehalose dihydrate in the group Var+Leu+Tre) revealed that the trends of the microparticulate particle flight distribution curves for both groups were similar. That is, regardless of whether mannitol or trehalose dihydrate was selected as the primary pharmacologically acceptable excipient (e.g., 84% in both groups), the dry powders prepared according to both groups exhibited similar aerosol properties.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A pharmaceutical composition for dry powder inhalation comprising:
an active ingredient, comprising an active compound with a molecular weight of less than 1000 Daltons; and
a pharmacologically acceptable excipient, comprising amino acid, sugar, sugar alcohol, polyol, phospholipid, or a combination thereof, wherein a weight ratio of the pharmacologically acceptable excipient and the active ingredient is from 1:1 to 99:1.

2. The pharmaceutical composition of claim 1, wherein a weight percentage of the active ingredient is from 1% to 50% and a weight percentage of the pharmacologically acceptable excipient is from 50% to 99% based on 100% by weight of the active ingredient and the pharmacologically acceptable excipient.

3. The pharmaceutical composition of claim 1, wherein the active compound comprises organic compound.

4. The pharmaceutical composition of claim 1, wherein the amino acid comprises glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline or a combination thereof.

5. The pharmaceutical composition of claim 1, wherein the sugar comprises disaccharide or polysaccharide.

6. The pharmaceutical composition of claim 5, wherein the disaccharide comprises sucrose, trehalose, lactose, maltose, or a combination thereof.

7. The pharmaceutical composition of claim 5, wherein the polysaccharide comprises chitosan, chitosan salt, chitosan glutamate, hyaluronic acid, starch, or a combination thereof.

8. The pharmaceutical composition of claim 1, wherein the sugar alcohol comprises glucose alcohol, xylitol, sorbitol, maltitol, erythritol, lactitol, mannitol, or a combination thereof.

9. The pharmaceutical composition of claim 1, wherein the polyol comprises polyvinyl alcohol, polyethylene glycol, or a combination thereof.

10. The pharmaceutical composition of claim 1, wherein the phospholipid comprises dipalmitoyl phosphatidylcholine, distearoyl phosphatidyl choline or a combination thereof.

11. The pharmaceutical composition of claim 1, wherein the active ingredient and the pharmacologically acceptable excipient forms a microparticulate particle having a particle size of from 50 nm to 5 μm.

12. The pharmaceutical composition of claim 11, wherein the microparticulate particle is provided in a spherical-shaped form, a solid polyhedron form, or a combination thereof.

13. The pharmaceutical composition of claim 1, wherein the pharmacologically acceptable excipient comprises a first pharmacologically acceptable excipient and a second pharmacologically acceptable excipient different from the first pharmacologically acceptable excipient.

14. The pharmaceutical composition of claim 13, wherein a weight percentage of the first pharmacologically acceptable excipient and the second pharmacologically acceptable excipient is from 1:1 to 1:99 when the first pharmacologically acceptable excipient is the amino acid, and the second pharmacologically acceptable excipient is the sugar or the sugar alcohol.

15. A method of preparing a pharmaceutical composition for dry powder inhalation, comprising:
dissolving an active ingredient and a pharmacologically acceptable excipient in a solvent to form a mixture, wherein the active ingredient comprises an active compound with a molecular weight of less than 1000 Daltons, and the pharmacologically acceptable excipient comprises amino acid, sugar, sugar alcohol, polyol, phospholipid, or a combination thereof, wherein a weight ratio of the pharmacologically acceptable excipient and the active ingredient is from 1:1 to 99:1; and
spray drying the mixture to form a pharmaceutical composition in a microparticulate form.

16. The method of claim 15, wherein dissolving the active ingredient and the pharmacologically acceptable excipient comprises dissolving the active ingredient, a first pharmacologically acceptable excipient and a second pharmacologically acceptable excipient in the solvent, wherein the second pharmacologically acceptable excipient is different from the first pharmacologically acceptable excipient.

17. The method of claim 16, wherein a weight percentage of the first pharmacologically acceptable excipient and the second pharmacologically acceptable excipient is from 1:1 to 1:99 when the first pharmacologically acceptable excipient is the amino acid, and the second pharmacologically acceptable excipient is the sugar or the sugar alcohol.

18. The method of claim 15, wherein the solvent comprises water, ethanol, methanol, dichloromethane, ethyl acetate, acetonitrile, acetone, dimethyl sulfoxide, or a combination thereof.

19. The method of claim 15, wherein a weight percentage of the active ingredient and the pharmacologically acceptable excipient is from 0.2% to 3% based on 100% by weight of the mixture.

20. The method of claim 15, wherein spray drying the mixture is performed at an outlet temperature of from 35° C. to 110° C.
